(19) United States
(12) Patent Application Publication
(10) Pub. No.: US 2021/0313066 A1
(43) Pub. Date: Oct. 7, 2021

(54) SYSTEM AND METHOD FOR AUTOMATED HEALTH AND FITNESS ADVISEMENT

(71) Applicant: Robert Ahlroth CAPPS, Decatur, GA (US)

(72) Inventor: Robert Ahlroth CAPPS, Decatur, GA (US)

(21) Appl. No.: 17/224,110

(22) Filed: Apr. 6, 2021

Related U.S. Application Data
(60) Provisional application No. 63/005,697, filed on Apr. 6, 2020.

(51) Int. Cl. G16H 50/20; G16H 50/50; G16H 40/67; G16H 10/60; G16H 20/30; G16H 15/00; G16H 50/70; G16H 50/30; G06F 16/901; G06N 3/08; A61B 5/00; A61B 5/0205; A61B 5/021; A61B 5/024; A61B 5/11; A61B 5/145; A61B 5/08

(52) U.S. Cl. CPC G16H 50/20; A61B 2560/0257; G16H 40/67; G16H 10/60; G16H 20/30; G16H 15/00; G16H 50/70; G16H 50/30; G06F 16/9024; G06N 3/08; A61B 5/681; A61B 5/02055; A61B 5/021; A61B 5/02438; A61B 5/1112; A61B 5/14532; A61B 5/14542; A61B 5/0816; A61B 5/743; A61B 2562/0219; G16H 50/50

(57) ABSTRACT

A system for the generation and maintenance of a virtual assistant that facilitates a systematic and psychological approach to health-improvement and self-care. Via interaction with the assistant, patients interact to create personalized health-improvement plans and adapt based upon physiological measurements and artificial intelligence analyses of data collected from the patient via either a sensor or patient input. The system may dynamically improve health recommendations based upon patient response to interaction with the system. A computational method with statistical inference of biophysical parameters that define an artificial intelligence's internal biophysical simulation environment, which does not rely on an artificial neural network. The corresponding method relies on an internal biophysical network that computationally replicates the patient's unique, health-relevant physiological processes, and uses psychological techniques to encourage improvement of the patient's health.

### CROSS-REFERENCE TO RELATED APPLICATIONS

To the full extent permitted by law, the present U.S. Non-Provisional Patent Application hereby claims priority to and the full benefit of, U.S. Provisional Application No. 63/005,697, filed on Apr. 6, 2020, entitled “Artificial Intelligence System for Automated Health Advertisement” which is incorporated herein by reference in its entirety.

### FIELD OF THE DISCLOSURE

The present disclosure is directed to a computational methodology and processes for advising an individual with regard to their health. More specifically, the present disclosure relates to a machine learning and artificial intelligence (AI) system for automatically advising patients to improve health, and reinforcing those advisements by sending personalized notifications to users/patients who can interact with the system via a specialized graphical user interface.

### BACKGROUND

Obesity, and the lack of general physical fitness, is a major individual and public health concern in the United States and throughout the world. In the United States alone, approximately more than a third of the adult population are considered obese and another third are considered overweight. Typical treatments of obesity and lack of physical fitness, as well as other weight-related and fitness disorders generally involve a multi-faceted approach. A diet may be furnished or recommended by a dietician or other healthcare provider in addition to a regimented exercise program. Behavioral health recommendations alone can be sufficient treatment in some instances. Moreover, behavioral advisement can be useful as a supplemental treatment in addition to invasive medical interventions in cases of severe illness or other medical conditions. In order to permanently impact a patient's wellbeing and longevity, these interventions commonly require significant and even permanent lifestyle modification. Relapse into unhealthy diet or lifestyle choices can degrade or diminish potential health benefits of any treatment—behavioral or otherwise. Especially in adults, the challenge of implementing and maintaining healthy lifestyle choices is often extremely difficult. Thus, even temporary recovery from obesity is uncommon—permanent recovery is practically unheard of.

The main goal of obesity management may be simply reducing the amount of adipose tissue (fat) in the patient. Other goals for the improvement of overall health may include increasing stamina during exercise, lowering blood pressure, decreasing cholesterol, lowering resting heart rate, decreasing sheer force upon vascular systems, increasing strength, the like and/or combinations thereof. Whether to motivate subjects, to enforce compliance or to troubleshoot/customize diets, it may be useful and important to have a means to track and trend overall progress toward increasing health and lifestyle goals, as well as physical benefits that may be commonly visible to the patient during a health and fitness intervention.

Individuals approaching peak physical fitness, such as athletes, and are often actively engaged in structured sports activities. While athletes, and other individuals whose lifestyle match their desires as it relates to their own physical fitness, may make sufficient choices and possess sufficient motivation to increase their physical health, they may struggle with making data-driven decisions about how best to optimize their biochemical and physical condition. While they may make healthy and wise decisions about how best to reach their fitness or health goals, they may lack insight as to which activities, diets, etc. may improve, or simply maintain, their healthy body condition. Moreover, retired athletes disproportionately (compared to the general populace) tend to struggle with adjusting to a less active lifestyle—and thus are prone to obesity and related health conditions.

Many devices currently exist which track, log, and inform a wearer of the device as to the physical activities which they participate. These devices may also measure certain biological information and inform their users' thereof. Beginning with rudimentary techniques, such as logging exercises into a journal, devices such as pedometers (which may crudely measure steps taken and/or distance walked/jogged) began to become popular in the second half of the 20th century. These devices have significantly matured to include heart rate, GPS, blood oxygen, and numerous other sensors to craft an ever-more-accurate picture of the physical exercises their wearers may engage over the course of days, weeks, months, and years. These devices now account for billions of dollars in annual sales, and nearly half a billion in annual devices shipped. In addition to simply taking any given measurement, these devices often interact with a platform, or may store a platform on the device, in order to generate reports as to progress along various metrics. Given that these sophisticated devices are now readily available to individuals for use in their daily life, the data gathered may be used by the individuals or may be interpreted by those caring for those individuals in a healthcare or fitness setting. However, while a user may be able to set a goal for achieving a certain regimen of exercise over a specific time period (e.g., 30 minutes of intense exercise 5 times a week), these devices, along with the platforms they may interact with, either lack the ability to provide advisement, encouragement, and reinforcement for achieving fitness goals, or require interaction with a third party “big data” or “supercomputing” system in order to provide this type of advisement to individuals in a highly relevant and user-influencing way.

It should also be noted that while devices which track the health and fitness progress of an individual through either generic or personalized plans may offer a user goals, plans, motivations, the like and/or combinations thereof, they possess serious shortfalls in the way of personalized motivation, needs assessment, and even risk factors. For instance, few offer motivation based on a plurality of psychological techniques known to influence behavior. Even those that may base a performance plan and goal setting upon a single psychological principal (e.g., providing a satisfying closure to a circle once a goal has been met), none are known to adapt to whether such a psychological influencing technique is helpful to a specific end user. So while a large portion of the population may be influenced by a specific psychological principal as it applied to following a performance plan or achieving goals, others who are not may only find that out about themselves after purchasing and subsequently giving up on using that motivational technique. While that user may purchase another device or subscribe to a different platform in order to better motivate themselves, without psychological insight into this personality trait, they may instead simply think of their failure as a moral or personal failing and simply give up. While this may do harm in-and-of-itself, other serious harms may occur should a smart health device or platform fail to perform assessment over the patient's, individual's, or end user's fitness status or health conditions. For instance, a smart fitness device or platform may fail to account for possible negative outcomes due to high physical stress activities on those who are unhealthy in various ways. In so doing, a smart fitness device may even have grave consequences to an end user who may suffer an acute physical event as a direct result of the encouragement to continue, despite what may be obvious physiological and/or physical risks to health.

Physicians, physiologists, personal trainers, dieticians and other individuals concerned with patient and client health have long sought to understand the mechanisms underlying human behavior, introspection, motivation and learning. Many of the obstacles related to increasing the health and longevity of patients relates to personal behavior, which may be able to be influenced through education and motivating techniques in order to achieve a lasting impact on a patient's lifetime health.

In one attempt to influence patient behavior, behavioral conditioning (i.e., “conditioning”) is a fundamental principle of modern learning theory, which can be summarized as any learning procedure that involves behavioral modification through the presentation of stimuli. Conditioning may often refer to a set of experimental procedures that attempt to modify (e.g., strengthen/weaken) internal associations between the presented stimuli, environmental cues, and specific behaviors. The concept of behavioral conditioning plays an important role in the semantic structure of neurofunctional theory.

In a branch of behavioral conditioning, behavioral reinforcement, or “reinforcement”, is another important concept to the influence of patient behavior. Reinforcement is a learning process that occurs when an experimental subject receives a rewarding stimulus as a consequence of performing a specific action, thereby increasing the probability that the subject will repeat that specific behavior. “Reinforcement learning” (RL) is then the process of repeated reinforcement to induce behavioral modification by strengthening or weakening (i.e., conditioning) the subject's internal reward-behavior associations.

In yet another branch, the development of cognitive-behavioral therapy (CBT) is possibly the longest-lasting applied psychological technique. CBT is a common type of psychological intervention that involves challenging and altering unhelpful thoughts, cognitive distortions, and behaviors to improve mental health.

Generally speaking, in order to influence a patient in a manner that is safe and likely to influence long-term behavior in a positive way, a health and fitness intervention must do so on both a physiological and a psychological basis. By implementing machine learning, in combination with biophysical monitoring, diet management, behavioral conditioning, cognitive-behavioral therapy, and positive reinforcement, an artificial intelligence system and method of healthcare advisement may be developed to both advise and encourage healthy and safe habits while monitoring and encouraging healthy living.

Therefore, a need exists for a health and fitness device, program, platform, or system/combination thereof which has the capability to monitor one or more of a plurality of biophysical metrics and exercise information in real-time, calculate, based on probability, how best to advise a patient with respect to their overall health and fitness, and encourage the patient in an effective way through use of machine learning and artificial intelligence. This long felt, but unresolved need must be met in order to provide more effective means to engage patients with these devices, programs, platforms, systems and combinations thereof.

The instant disclosure may be designed to address at least certain aspects of the problems or needs discussed above by providing a health and fitness device, program, platform, or system/combination thereof which has the capability to monitor one or more of a plurality of biophysical metrics and exercise information in real-time, calculate—based on probability—how best to advise a patient with respect to their overall health and fitness, and encourage the patient in an effective way through use of machine learning and artificial intelligence.

### SUMMARY

The present disclosure may solve the aforementioned limitations of the currently available storage and organization devices, like closet organization devices, by providing a health and fitness device, program, platform, or system/combination thereof which has the capability to monitor one or more of a plurality of biophysical metrics and exercise information in real-time, calculate, based on probability, how best to advise a patient with respect to their overall health and fitness, and encourage the patient in an effective way through use of machine learning and artificial intelligence

Accordingly, in one aspect, the present disclosure embraces an artificial intelligence virtual assistant that facilitates a systematic approach to health-improvement (self-care). Users may interact with the virtual assistant to create personalized health-improvement plans. The system may then adapt to physiological measurements (e.g., heart-rate from a wearable activity monitor), which the virtual assistant could then use, via machine learning, to dynamically improve health recommendations. A computational method involves statistical inference of biophysical parameters that define the system's internal biophysical simulation environment. In other words, some measurements may be obtained, some measurements may be monitored, and others may be inferred based upon computations and obtained and/or monitored data. Then a full picture of the health and fitness of an individual may be created and advice may be given to the individual based upon the inputs, measurements, and calculations. Importantly the system of the disclosure may offer this advice and assemble these inferences without needing to rely on an artificial neural network, which may be a key distinguishing of the system of the disclosure. Instead, the machine learning system may rely on an internal biophysical network that computationally replicates the user's unique, health-relevant physiological processes.

In one aspect, the system of the disclosure may utilize a biophysics simulation environment. In this aspect, an internal (i.e., not cloud computing or “big data”) biophysical simulation environment that produces simulated biophysical and/or physiological output (e.g., electrophysiological, neuronal activity). This aspect may enable physiological interpretation of the system of the disclosure's output via artificial intelligence and machine learning. The biophysical model of this aspect may also quantify a variety of physiological processes (e.g., neuronal, muscular, metabolic) by combining several biophysical models, from which physiological simulations are generated. By assembling the system in this way, it may become completely modular. Each of the modules (simulated physiological processes) may be understood to be substituted for user-recorded physiology data or measured data, which would enhance predictive accuracy of the biophysical simulation. Through use of this aspect, namely the biophysics simulation environment, the system may also enable the production of health, fitness, and wellness recommendations with or without user-recorded data. By way of example and not limitation, even if a user does not have a wearable fitness tracker, or if the user has temporarily misplaced it, the biophysics simulation environment through use of machine learning and artificial intelligence, the virtual assistant may prompt the user to enter periodic health updates, which could be used to produce wellness recommendations, independent of any physiological recordings, albeit at the possible cost of accuracy. In a potentially ideal embodiment of this aspect of the disclosure, the biophysics simulation environment may approximate medical-grade precision at reduced cost by estimating the biophysical properties of health-relevant physiology.

In another aspect, the system of the disclosure may utilize a health network graph. In this aspect, a user interface may include a “Health Network Graph”, which may be an interactive network diagram or web which may include organs or biological systems which may be represented as nodes in the graph. Physiological connections between organs or biological systems may be represented as connections between the nodes, or as edges of the graph. By way of example and not limitation, the health network graph of the system of the disclosure may represent the heart and lungs as nodes, and the pulmonary artery as a connection between the nodes of the heart and lungs. Users may interact with the Health Network Graph to view detailed information about each physiological structure, including health-relevant statistics and estimated biophysical measurements. These biophysical measurements may be obtained through user entry (e.g., height and weight), through measurements (e.g., via a fitness wearable), and/or computed from individual user data using the biophysics simulation, artificial intelligence, and machine learning. By visualizing health relevant organs and systems in this way, the system of the disclosure, namely the Health Network Graph as represented in a graphical user interface, may enable users to better understand the quantitative reasoning behind the virtual assistant's recommendations. This understanding alone may encourage optimal motivation and desire to accomplish the goals and plans presented by the virtual assistant.

In an exemplary embodiment of the system of the disclosure, the virtual assistant having artificial intelligence and machine learning may combine various features of well-known computational techniques to select an optimal series of actions that would enable the user to accomplish a specified health improvement goal. When an optimal series of actions is determined, the result may be displayed as a “wellness recommendation” to help accomplish the personalized health goal. The artificial intelligence and machine learning architecture may be essentially an agent-based “biophysical” recurrent neural network, in which individual network units replicate temporal dynamics of the physiological processes. Examples of such computational techniques and calculations are further described in the detailed description below. Action selection may be implemented as an iterative stochastic optimization process that begins with exploration. The system of the disclosure may “explore” possible action sequences by simulating the physiological consequences of each action within the internal biophysical network, recording each action and the corresponding network configuration. The biophysical parameters for each network component may then be sampled from a statistical distribution, which is defined by the average and standard error computed from user data. Optimal actions may be selected by maximum likelihood estimation, using a cost function that incorporates the user's current health, the specified goal, and the corresponding simulated physiological output. The algorithm may meet convergence criteria when an optimal action sequence is found that would accomplish the user's health goal within a user-specified timeframe, or if the network space was explored without finding a working solution. If the network space was explored without finding a working solution, the system of the disclosure may then offer the user the choice to either extend the time to complete the goal or specify a less ambitious health goal.

The foregoing illustrative summary, as well as other exemplary objectives and/or advantages of the disclosure, and the manner in which the same are accomplished, are further explained within the following detailed description and its accompanying drawings.

### BRIEF DESCRIPTION OF THE DRAWINGS

The present disclosure will be better understood by reading the Detailed Description with reference to the accompanying drawings, which are not necessarily drawn to scale, and in which like reference numerals denote similar structure and refer to like elements throughout, and in which:

FIG. 1 is a perspective drawing of a person exercising while wearing a smart health and fitness device;

FIG. 2 is a is a block diagram of a computer system of the present disclosure;

FIG. 3 is a block diagram of a communications system implemented by the computer system in FIG. 2;

FIG. 4 is a block diagram of an exemplary embodiment of a health network graph of the disclosure;

FIG. 5 is a flowchart of an exemplary embodiment of a method of generating a healthcare advisement for a patient;

FIG. 6 is a first exemplary user interface for a user generation of a healthcare advisement of a patient; and

FIG. 7 is a second exemplary user interface for a user generation of a healthcare advisement of a patient.

It is to be noted that the drawings presented are intended solely for the purpose of illustration and that they are, therefore, neither desired nor intended to limit the disclosure to any or all of the exact details of construction shown, except insofar as they may be deemed essential to the claimed disclosure.

### DETAILED DESCRIPTION

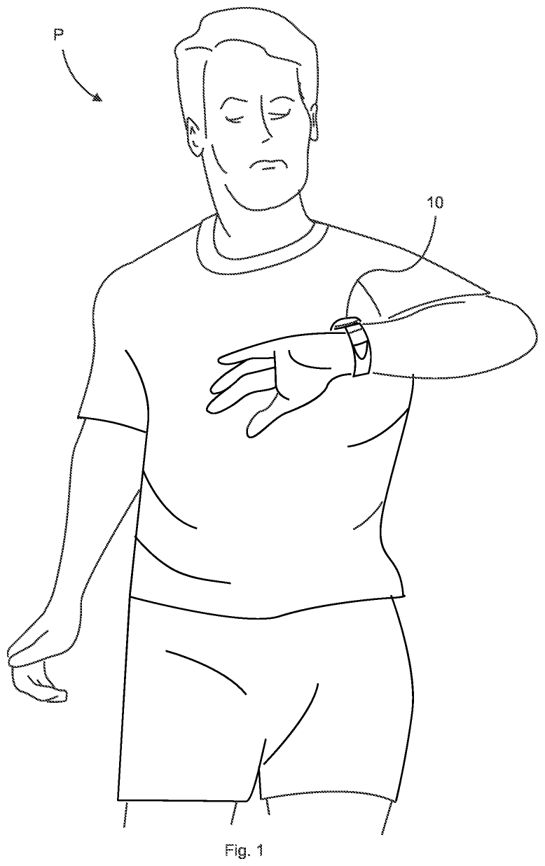

Referring now to FIGS. 1-7, in describing the exemplary embodiments of the present disclosure, specific terminology is employed for the sake of clarity. The present disclosure, however, is not intended to be limited to the specific terminology so selected, and it is to be understood that each specific element includes all technical equivalents that operate in a similar manner to accomplish similar functions. Embodiments of the claims may, however, be embodied in many different forms and should not be construed to be limited to the embodiments set forth herein. The examples set forth herein are non-limiting examples and are merely examples among other possible examples.

The present disclosure solves the aforementioned limitations of the currently available devices, platforms, systems and methods of encouraging healthy living and physical fitness by providing a health network graph capable of visually presenting a biosimulation of a patient system of a patient's organs and body, measuring biophysical conditions of one or more of a plurality of metrics used to measure the conditions of a patient's organs and body, calculating measurements of biophysical conditions of those unmeasured, and returning an advisement to a patient with respect to various diets, calorie intakes, physical activities, motivations, workout plans, sleep plans, breathing exercises, stretches, the like and/or combinations thereof which may influence improvements across various patient organs and physiological symptoms. By using machine learning to predict the outcome of an advisement on both a physiological and psychological level, the present disclosure and its corresponding systems and methods may overcome an inability to positively influence all individuals in current devices, systems, platforms, programs, and the like by generating, adapting, and machine learning with regard to how a patient responds to stimuli, such as an advisement to a healthy or fitness-oriented activity or behavior.

Referring now specifically to FIG. 1, therein illustrated is patient P, who is a jogger wearing computing device 10, which may be understood as a wearable health and fitness tracking device, or smart health wearable. As will be understood below, computing device 10 may feature a variety of computing parts, components, and functions, including but not limited to biometric detection, visual display, storage, computing, networking, input, and audio-playback. With regard to biometric or other information detection, it should be understood by those skilled in the art that any and all measures relevant to human health and fitness quantification may be a feature of computing device 10. These include but are not limited to location using GPS, heart rate, calorie intake logging, blood pressure, body composition, barometric sensing, altimetry, pedometer, sleep logging, blood oxygen, body temperature, ambient temperature, gyroscope, accelerometer, blood glucose, respiration rate, weight, height, the like and/or combinations thereof. These measurements may be obtained via a sensor installed in or on computing device 10 such as I/O devices 212, otherwise physically connected to computing device 10, wirelessly connected to computing device 10, networked to computing device 10 via network device 210, or entered into computing device 10 via an input such as a touchscreen, keyboard, or camera with optical character recognition. It should be noted that while computing device 10 is illustrated herein as resembling a smart wristwatch, the disclosure is not so limited. Computing device 10 can be configured in an unlimited number of configurations and need not be wearable. By way of example, computing device 10 may be constructed as a smart phone, an armband, a leg band, a waist band, a headset, a head band, a ring, a piercing, a necklace, a neckband, inseparable with or placed into an article of clothing or shoe, a stationary device, the like and/or combinations thereof. Because computing device 10 may contain a plurality of sensitive information about patient P, it may further feature security settings which enable the additional capability of authentication via biometrics. These include but are not limited to fingerprint, facial, voice, iris, and retinal recognition, as well as keystroke dynamics and/or signature recognition. Computing device 10 may also include a plurality of devices either networked or otherwise connected to obtain health and fitness information relevant to patient P health and fitness.

As will be appreciated by one of skill in the art, the present disclosure may be embodied as a method, data processing system, or computer program product. Accordingly, the present disclosure may take the form of an entirely hardware embodiment, entirely software embodiment or an embodiment combining software and hardware aspects. Furthermore, the present disclosure may take the form of a computer program product on a computer-readable storage medium having computer-readable program code means embodied in the medium. Any suitable computer readable medium may be utilized, including hard disks, flash storage, ROM, RAM, CD-ROMs, electrical, optical, magnetic storage devices and the like.

The present disclosure is described below with reference to flowchart illustrations of methods, apparatus (systems) and computer program products according to embodiments of the present disclosure. It will be understood that each block or step of the flowchart illustrations, and combinations of blocks or steps in the flowchart illustrations, can be implemented by computer program instructions or operations. These computer program instructions or operations may be loaded onto a general-purpose computer, special purpose computer, or other programmable data processing apparatus to produce a machine, such that the instructions or operations, which execute on the computer or other programmable data processing apparatus, create means for implementing the functions specified in the flowchart block or blocks/step or steps.

These computer program instructions or operations may also be stored in a computer-usable memory that can direct a computer or other programmable data processing apparatus to function in a particular manner, such that the instructions or operations stored in the computer-usable memory produce an article of manufacture including instruction means which implement the function specified in the flowchart block or blocks/step or steps. The computer program instructions or operations may also be loaded onto a computer or other programmable data processing apparatus (processor) to cause a series of operational steps to be performed on the computer or other programmable apparatus (processor) to produce a computer implemented process such that the instructions or operations which execute on the computer or other programmable apparatus (processor) provide steps for implementing the functions specified in the flowchart block or blocks/step or steps.

Accordingly, blocks or steps of the flowchart illustrations support combinations of means for performing the specified functions, combinations of steps for performing the specified functions, and program instruction means for performing the specified functions. It should also be understood that each block or step of the flowchart illustrations, and combinations of blocks or steps in the flowchart illustrations, can be implemented by special purpose hardware-based computer systems, which perform the specified functions or steps, or combinations of special purpose hardware and computer instructions or operations.

Computer programming for implementing the present disclosure may be written in various programming languages, database languages, and the like. However, it is understood that other source or object-oriented programming languages, and other conventional programming language may be utilized without departing from the spirit and intent of the present disclosure.

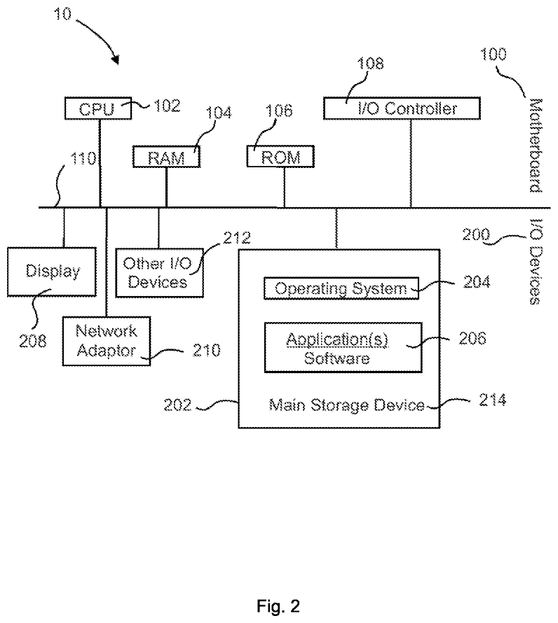

Referring now to FIG. 2, there is illustrated a block diagram of a computing system 10 that provides a suitable environment for implementing embodiments of the present disclosure. The computer architecture shown in FIG. 2 is divided into two parts—motherboard 100 and the input/output (I/O) devices 200. Motherboard 100 preferably includes subsystems and/or processor(s) to execute instructions such as central processing unit (CPU) 102, a memory device, such as random-access memory (RAM) 104, input/output (I/O) controller 108, and a memory device such as read-only memory (ROM) 106, also known as firmware, which are interconnected by bus 110. A basic input output system (BIOS) containing the basic routines that help to transfer information between elements within the subsystems of the computer is preferably stored in ROM 106, or operably disposed in RAM 104. Computing system 10 further preferably includes I/O devices 202, such as main storage device 214 for storing operating system 204 and instructions or application program(s) 206, and display 208 for visual output, and other I/O devices 212 as appropriate. Main storage device 214 preferably is connected to CPU 102 through a main storage controller (represented as 108) connected to bus 110. Network adapter 210 allows the computer system to send and receive data through communication devices or any other network adapter capable of transmitting and receiving data over a communications link that is either a wired, optical, or wireless data pathway. It is recognized herein that central processing unit (CPU) 102 performs instructions, operations or commands stored in ROM 106 or RAM 104.

Processor 102 may, for example, be embodied as various means including one or more microprocessors with accompanying digital signal processor(s), one or more processor(s) without an accompanying digital signal processor, one or more coprocessors, one or more multi-core processors, one or more controllers, processing circuitry, one or more computers, various other processing elements including integrated circuits such as, for example, an ASIC (application specific integrated circuit) or FPGA (field programmable gate array), or some combination thereof. Accordingly, although illustrated in FIG. 2 as a single processor, in some embodiments, processor 102 comprises a plurality of processors. The plurality of processors may be embodied on a single computing device or may be distributed across a plurality of computing devices collectively configured to function as computing device 10. The plurality of processors may be in operative communication with each other and may be collectively configured to perform one or more functionalities of computing device 10 as described herein. In an example embodiment, processor 102 is configured to execute instructions stored in memory 104, 106 or otherwise accessible to processor 102. These instructions, when executed by processor 102, may cause the computing device 10 to perform one or more of the functionalities of computing device 10 as described herein.

Whether configured by hardware, firmware/software methods, or by a combination thereof, processor 102 may comprise an entity capable of performing operations according to embodiments of the present invention while configured accordingly. Thus, for example, when processor 102 is embodied as an ASIC, FPGA or the like, processor 102 may comprise specifically configured hardware for conducting one or more operations described herein. As another example, when processor 102 is embodied as an executor of instructions, such as may be stored in memory 104, 106, the instructions may specifically configure processor 102 to perform one or more algorithms and operations described herein.

The plurality of memory components 104, 106 may be embodied on a single computing device 10 or distributed across a plurality of computing devices. In various embodiments, memory may comprise, for example, a hard disk, random access memory, cache memory, flash memory, a compact disc read only memory (CD-ROM), digital versatile disc read only memory (DVD-ROM), an optical disc, circuitry configured to store information, or some combination thereof. Memory 104, 106 may be configured to store information, data, applications, instructions, or the like for enabling the computing device 10 to carry out various functions in accordance with example embodiments discussed herein. For example, in at least some embodiments, memory 104, 106 is configured to buffer input data for processing by processor 102. Additionally, or alternatively, in at least some embodiments, memory 104, 106 may be configured to store program instructions for execution by processor 102. Memory 104, 106 may store information in the form of static and/or dynamic information. This stored information may be stored and/or used by the computing device 10 during the course of performing its functionalities.

Many other devices or subsystems or other I/O devices 212 may be connected in a similar manner, including but not limited to, devices such as microphone, speakers, flash drive, CD-ROM player, DVD player, printer, main storage device 214, such as hard drive, and/or modem each connected via an I/O adapter. Also, although preferred, it is not necessary for all of the devices shown in FIG. 2 to be present to practice the present disclosure, as discussed below. Furthermore, the devices and subsystems may be interconnected in different configurations from that shown in FIG. 2, or may be based on optical or gate arrays, or some combination of these elements that is capable of responding to and executing instructions or operations. The operation of a computer system such as that shown in FIG. 2 is readily known in the art and is not discussed in further detail in this application, so as not to overcomplicate the present discussion.

In some embodiments, some or all of the functionality or steps may be performed by processor 102. In this regard, the example processes and algorithms discussed herein can be performed by at least one processor 102. For example, non-transitory computer readable storage media can be configured to store firmware, one or more application programs, and/or other software, which include instructions and other computer-readable program code portions that can be executed to control processors of the components of system 201 to implement various operations, including the examples shown above. As such, a series of computer-readable program code portions may be embodied in one or more computer program products and can be used, with a computing device, server, and/or other programmable apparatus, to produce the machine-implemented processes discussed herein.

Any such computer program instructions and/or other type of code may be loaded onto a computer, processor or other programmable apparatuses circuitry to produce a machine, such that the computer, processor or other programmable circuitry that executes the code may be the means for implementing various functions, including those described herein.

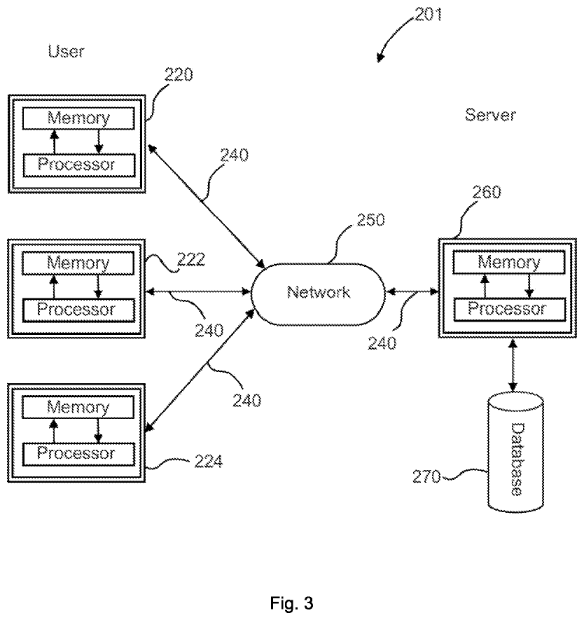

Referring now to FIG. 3, there is illustrated a diagram depicting an exemplary system 201 in which concepts consistent with the present disclosure may be implemented or performed. Examples of each element within the communication system 201 of FIG. 3 are broadly described above with respect to FIG. 2. In particular, the server system 260 and user system 220 have attributes similar to computer system 10 of FIG. 2 and illustrate one possible implementation of computer system 10. Communication system 201 preferably includes one or more user systems 220, 222, 224, one or more server system 260, and network 250, which could be, for example, the Internet, public network, private network or cloud. User systems 220-224 each preferably include a computer-readable medium, such as random-access memory, coupled to a processor. The processor, CPU 102, executes program instructions or operations stored in memory. Communication system 201 typically includes one or more user system 220. For example, user system 220 may include one or more general-purpose computers (e.g., personal computers), one or more special purpose computers (e.g., devices specifically programmed to communicate with each other and/or the server system 260), a workstation, a server, a device, a digital assistant or a “smart” cellular telephone or pager, a digital camera, a component, other equipment, or some combination of these elements that is capable of responding to and executing instructions or operations.

Similar to user system 220, server system 260 preferably includes a computer-readable medium, such as random-access memory, coupled to a processor. The processor executes program instructions stored in memory. Server system 260 may also include a number of additional external or internal devices, such as, without limitation, a mouse, a CD-ROM, a keyboard, a display, a storage device and other attributes similar to computer system 10 of FIG. 2. Server system 260 may additionally include a secondary storage element, such as database 270 for storage of data and information. Server system 260, although depicted as a single computer system, may be implemented as a network of computer processors. Memory in server system 260 contains one or more executable steps, program(s), algorithm(s), or application(s) 206 (shown in FIG. 1). For example, the server system 260 may include a web server, information server, application server, one or more general-purpose computers (e.g., personal computers), one or more special purpose computers (e.g., devices specifically programmed to communicate with each other), a workstation or other equipment, or some combination of these elements that is capable of responding to and executing instructions or operations.

System 201 is capable of delivering and exchanging data between user system 220 and a server system 260 through communications link 240 and/or network 250. Through user system 220, users can preferably communicate over network 250 with each other user system 220, 222, 224, and with other systems and devices, such as server system 260, to electronically transmit, store, manipulate, and/or otherwise use data exchanged between the user system and the server system. Communications link 240 typically includes network 250 making a direct or indirect communication between the user system 220 and the server system 260, irrespective of physical separation. Examples of a network 250 include the Internet, cloud, analog or digital wired and wireless networks, radio, television, cable, satellite, and/or any other delivery mechanism for carrying and/or transmitting data or other information, such as to electronically transmit, store, manipulate, and/or otherwise modify data exchanged between the user system and the server system. The communications link 240 may include, for example, a wired, wireless, cable, optical or satellite communication system or other pathway. It is contemplated herein that RAM 104, main storage device 214, and database 270 may be referred to herein as storage device(s) or memory device(s).

The illustrations of FIGS. 2-3 as described herein are intended to provide a general understanding of the structure of various embodiments of computing devices operating in a networked fashion. The illustrations are not intended to serve as a complete description of all of the elements and features of apparatus, processors, and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

With respect to the above description then, it is to be realized that the optimum dimensional relationships, to include variations in size, materials, shape, form, position, function and manner of operation, assembly, type of biometric sensor, availability of feedback and reporting, and use, are intended to be encompassed by the present disclosure.

It is contemplated herein that any device used in the system of the disclosure may include a variety of overall sizes, functions, sensors, computing functionality and corresponding sizes for and of various parts, including but not limited to: servers, computing devices, sensors, communication devices and/or protocols, systems architecture, power source(s), user interfaces, the like and/or combinations thereof as well as various electronic components to accommodate different needs and/or functions. Furthermore, it is contemplated that due to variations in the human body among the population, that a variety of considerations may be considered in regard to the placement and availability of sensors and biofeedback. Yet still, though the inventor has contemplated multiple methods of selecting and biosimulating a virtual health and fitness recommendation assistant, the disclosure is not limited. Other means have been contemplated, and the disclosure not so limited. Various trade-offs may be considered when selecting the technology, sensors, communication protocols, user interfaces, the like and/or combinations thereof to deploy system of the disclosure. These may include favoring less invasive devices and/or sensors for general fitness assistance and more invasive devices and/or sensors for assisting individuals having health conditions needing more specific data (e.g. blood sugar readings via an implanted device for diabetic individuals). It is also contemplated that certain considerations and/or additional features of the present disclosure may improve the functionality. These may include augmented reality, heads-up display, haptic feedback, electroshock, the like, and/or combinations thereof. In regard to communication with other devices via a network, the device may communicate via any known or yet to be discovered protocol, including wired networking, fiber optic communication, satellite networks, wireless networking (i.e. WiFi), near field communication (e.g. Bluetooth® or NFC), the like or combinations thereof. The device may receive power from an outlet designed for consumer or commercial electronics, or may contain a battery which may or may not have the capability to re-charge.

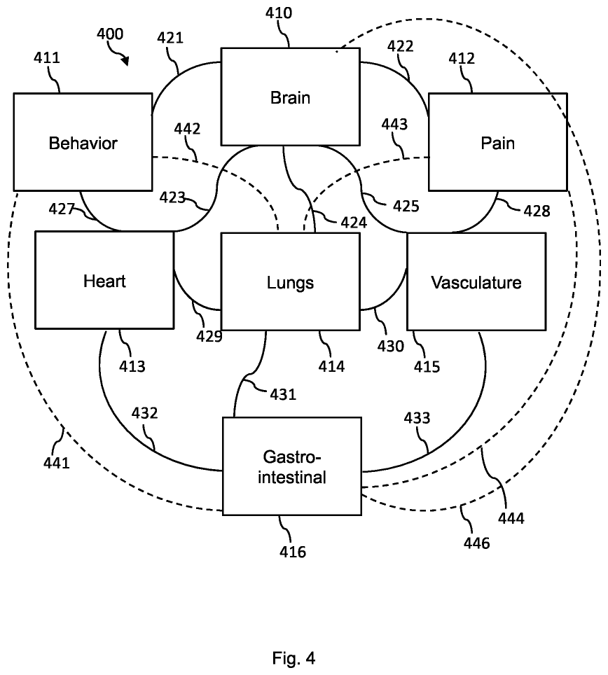

Referring now specifically to FIG. 4, therein illustrated is a block diagram of an exemplary embodiment of health network graph 400 of the disclosure. For reference, health network graph 400 may be both a visual and analytical representation of various systems and organs of a human body. The various organs and systems represented therein FIG. 4 are exemplary only, and are in no way exhaustive or limiting of the disclosure. Health network graph 400, as illustrated herein, contains the organs and systems of brain 410, behavior 411, pain sensation 412, heart 413, lungs 414, vasculature 415, and gastrointestinal 416, represented as blocks in the diagram. Solid and dotted lines of health network graph 400 may represent connections, either direct or indirect, respectively, by which one organ or system may influence another. These include the direct interactions and solid lines of connection between brain and behavior 421, connection between brain and pain sensation 422, connection between brain and heart 423, connection between brain and lungs 424, connection between brain and vasculature 425, connection between behavior and heart 427, connection between pain and vasculature 428, connection between heart and lungs 429, connection between vasculature and lungs 430, connection between lungs and gastrointestinal system 431, connection between heart and gastrointestinal system 432, and connection between vasculature and gastrointestinal system 433. The connections may be physical or causal and may be unidirectional or bi-directional. By way of example and not limitation, a pain stimulus may be sensed in the brain as well as influences on the brain may have a dulling and/or exaggerating effect on the sensation of pain, therefore this may be best understood as a direct relationship and is represented by a solid line. Indirect and dotted lines as represented herein may include connection between behavioral and gastrointestinal system 441, connection between behavioral and lungs 442, connection between pain and lungs 443, connection between pain and gastrointestinal 444, and connection between brain and gastrointestinal system 446.

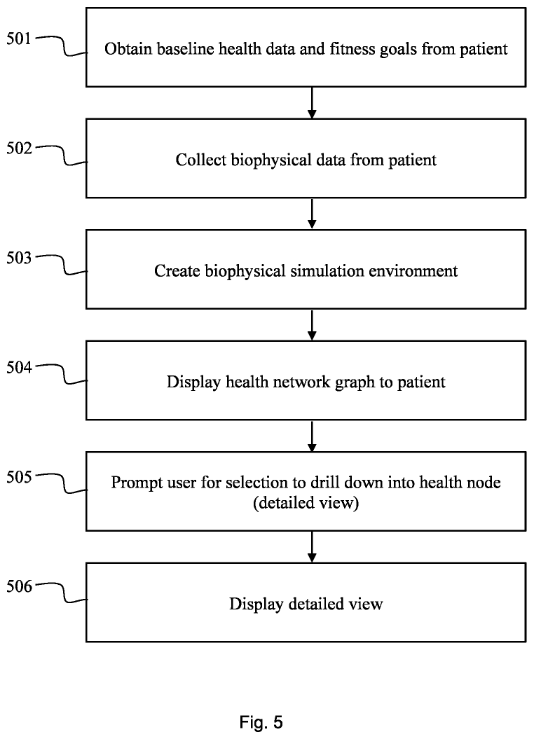

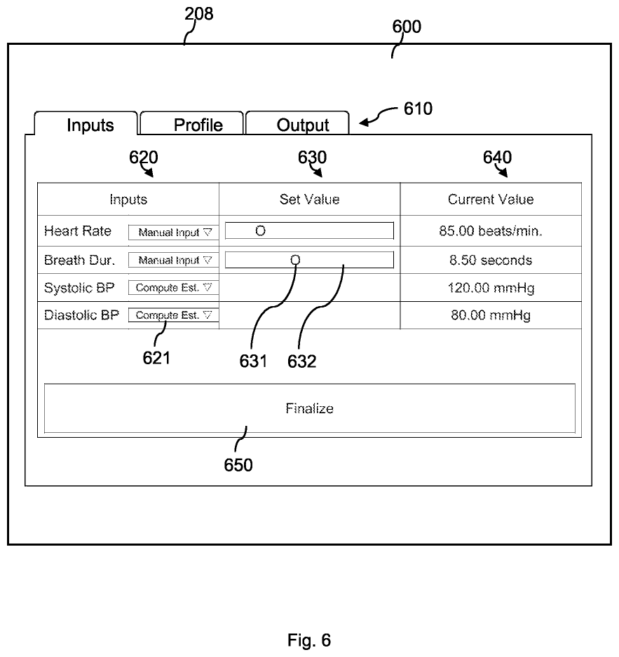

By way of example and not limitation, health network graph 400 may be used in at least the following ways: as a visual representation of patient P and patient P's overall wellness and as an organizational principal designed to train a virtual assistant using artificial intelligence and machine learning. With regard to the former, a visual representation using health network graph 400 may be presented to patient P via display 208, patient P may select a node (e.g., brain 410 or heart 413) for further exploration. Having tapped, clicked, or otherwise selected the node, further information may be provided to patient P which may explain a health or fitness condition of that node using quantitative information. This further information will be more readily apparent based on the descriptions related to FIGS. 5 and 6. With regard to the latter, using health network graph 400 as a basis, foundation, or principle behind organizing and training a virtual assistant using artificial intelligence and machine learning, by obtaining information from patient P during baseline intake, and through surveying and/or detecting health and fitness biological information via device 10, then by calculating further information about organs and system interactions (e.g. connection between brain and heart 423), a virtual assistant stored on device 10 may make further calculations in order to further determine, via probability and multiple biophysical simulations, the likely health and fitness of organs which are not constantly monitored.

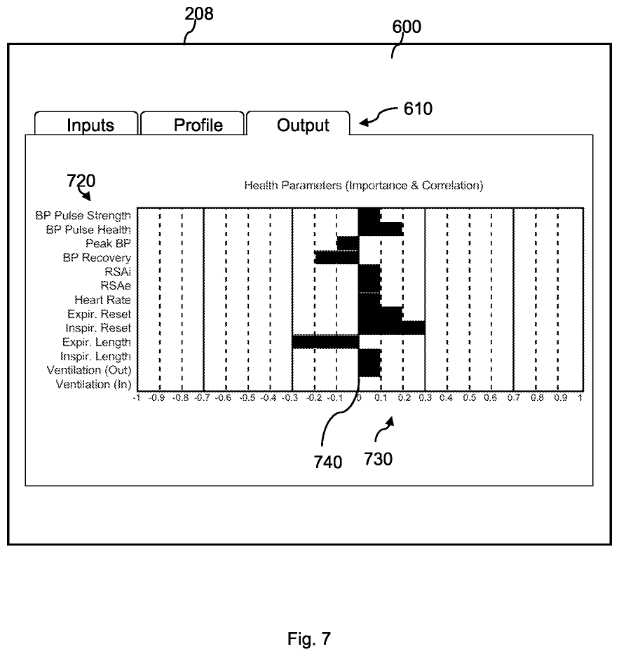

Referring now specifically to FIG. 5, therein illustrated is a flowchart of an exemplary embodiment of a method of generating a healthcare advisement for a patient. Starting at first method step 501, a user may be prompted to enter baseline health data and fitness goals into a system of the disclosure. Next, at second method step 502, information, specifically biophysical information, is collected by computing device 10 from patient P (e.g., heart rate). Then at third method step 503, a biophysical simulation environment is created by the system of the disclosure. At fourth method step 504, health network graph 400 is displayed to the patient. The user, namely patient P, is then prompted to select a health node to view in detailed mode (e.g., heart or cardiovascular system), at fifth method step 505. Finally, at sixth method step 506, a detailed view may be displayed to patient P on display 208, such as exemplary user interface 600. For the exemplary detailed view of exemplary user interface 600 see FIG. 7.

Referring now specifically to FIG. 6, therein illustrated is a first view of exemplary user interface 600 for a user generation of a healthcare advisement of a patient on display 208 of computing device 10. At the top of exemplary user interface 600 may be series of tabs 610. As illustrated herein, those tabs may include “Inputs”, “Profile”, and “Outputs”. As is further illustrated herein, of series of tabs 610, “Inputs” has been selected to reveal inputs column 620 having a series of biophysical measurements and input dropdown 621, values column 630 having value selector 631 on selector bar 632, current value column 640 and finalize button 650. By way of example and not limitation, patient P, having set up a virtual assistant using the biophysical simulation environment system of the disclosure, may then manually input heart rate and breath duration into device 10 by first selecting “Inputs” on series of tabs 610, then selecting the current heart rate by sliding value selector 631 along selector bar 632. Upon selection of heart rate, current value column 640 may then show, based on the selection of sliding value selector 631 along selector bar 632, what BPM patient P selected. In this case 85 BPM was selected. If this is incorrect, patient P may revise by again adjusting sliding value selector 631 along selector bar 632. Patient P may continue by selecting other variables which may include those in FIG. 6, or may include other values relevant to other organs or biological systems. When patient P is ready to learn more about calculations and simulations relevant to the selection chosen, patient P may hit “Finalize” on finalize button 650.

Referring now specifically to FIG. 7, therein illustrated is a second view of exemplary user interface 600 for a user generation of a healthcare advisement of a patient on display 208 of computing device 10. At the top of exemplary user interface 600 may be series of tabs 610. As illustrated herein, those tabs may include “Inputs”, “Profile”, and “Outputs”. As is further illustrated herein, of series of tabs 610, “Output” has been selected to reveal a chart, the chart having health and fitness category axis 720, value axis 730, and zero-value band 740. Along health and fitness category axis 720 may be a series of quantitative and qualitative assessments of patient P, based on inputs and/or calculations obtained on the previous screen (See FIG. 6). Included therein FIG. 7 along health and fitness category axis 720 are BP pulse strength, BP pulse health, peak BP, BP recovery, respiratory sinus arrythmia (inspiration and expiration), heart rate, expiration reset, inspiration reset, expiration length, inspiration length, and ventilation (out and in). Health and fitness category axis 720 may also be understood as a parameter distribution for a cardiovascular model. Each bar on user interface 600 at each of health and fitness category axis 720 positions may represent, via size, a magnitude relative to the distribution, and the sign (i.e., whether positive or negative) may indicate which other parameters any value is coordinated.

Given that the systems and methods of the disclosure require complex mathematics be applied to calculate, or to make inferences, regarding the unmeasured information about biological systems, many bioinformatic statistical techniques and calculations may be relevant to various implementations of the systems and methods of the disclosure. While the formulas provided herein may be highly relevant to the cardiovascular and neurological systems, and their relevant interactions, the disclosure is not so limited. Therefore, by way of example and not limitation, where initial respiration volume is R0, calculation of inspiration, expiration, and respiration may be obtained via the following calculations: inspiration:

$$R 1 ( t , R ( t ) ) = exp ( \frac{\sqrt{R ( t )} - B}{{τ}_{1}} ) , \\ R 2 ( t , R ( t ) ) = \frac{exp ( - \sqrt{R ( t )} - B )}{3 {τ}_{2}} , and$$

$$\frac{d R ( t )}{d t} = R 0 + A {e}^{- {( R 1 ( t ) - R 2 ( t ) )}^{2}} ,$$

respectively. Vagal activation, sympathetic activation, heart phase, initial R-R interval, and RRI Rate may be obtained via the following calculations:

$${Θ}_{V} = \frac{v}{1 - {e}^{- R ( t )}} , \\ {Θ}_{S} = \frac{S}{1 - {e}^{R ( t )}} , {ϕ}_{H} ( t ) , \\ {RRI}_{0} = \frac{60 sec}{H {R}_{0}} , and$$

$$\frac{( d {ϕ}_{H} ( t ) )}{d t} = {RRI}_{0} + {Θ}_{V} ( \frac{d R ( t )}{d t} ) + {Θ}_{S} ( \frac{d R ( t )}{d t} ) ,$$

respectively. Maximum likelihood may be calculated where Θ*=argmax_{Θ}Σⱼ₌₁^{m} L(t, x; θⱼ) where L(t, x, θ)=exp{−(x(t)−f(t, θ)²} is the conditional likelihood of the sample (t, x), given the true parameters θ. Through use of Hessian-approximated Covariance Matrix of

$${Cov}^{*} = {[ - ( \frac{1}{m} ) {Σ}_{j = 1}^{m} \ddot{∇} L ( t , x ; {Θ}_{m} ) ]}^{- 1} ,$$

where \ddot{∇}L(t, x; θₘ) is the Hessian matrix (second-order partial derivative) of the likelihood. An appetite model may include the following system of coupled, nonlinear equations:

$${τ}_{E} \cdot \frac{d E ( t )}{d t} = H ( t ) {α}_{E} \cdot {E ( t )}^{3} - S ( t ) + {I}_{M} ( t ) , \\ {τ}_{H} \cdot \frac{d H ( t )}{d t} = {α}_{H} - {β}_{H} \cdot {E ( t )}^{2} - H ( t ) + {I}_{H} ( t ) , and$$

$${τ}_{S} \cdot \frac{d S ( t )}{d t} = 1 e - 3 ( {α}_{S} \cdot E ( t ) - {V}_{R} ) - {β}_{H} \cdot S ( t ) + {I}_{E , H} ( t ) - {I}_{S} ( t ) \text{)}$$

where appetite is described as a combination of three dynamical variables: “eating” E(t), “hunger” H(t), and “satiety” S(t).

In a potentially preferred exemplary embodiment of the system of the disclosure, the virtual assistant having artificial intelligence and machine learning may display, or cause to be displayed, various health advisements on display 208 of computing device 10. It may assemble these advisements by combining various features of well-known computational techniques to select an optimal series of actions that would enable the user to accomplish a specified health improvement goal. When an optimal series of actions is determined, by performing these calculations via processor 102 based on health information of patient P stored as data on main storage device 214, the result may be displayed on display 208 as a “wellness recommendation” to help accomplish the personalized health goal. The artificial intelligence and machine learning architecture may be essentially an agent-based “biophysical” recurrent neural network, in which individual network units replicate temporal dynamics of the physiological processes. Examples of such computational techniques and calculations, such as those above, may be performed on processor 102 of computing device 10. Action selection may be implemented as an iterative stochastic optimization process that begins with exploration. The system of the disclosure may “explore” possible action sequences by simulating the physiological consequences of each action within the internal biophysical network, recording each action and the corresponding network configuration. The biophysical parameters for each network component may then be sampled from a statistical distribution, which is defined by the average and standard error computed from user data. Optimal actions may be selected by maximum likelihood estimation, using a cost function that incorporates the user's current health, the specified goal, and the corresponding simulated physiological output. The algorithm may meet convergence criteria when an optimal action sequence is found that would accomplish the user's health goal within a user-specified timeframe, or if the network space was explored without finding a working solution. If the network space was explored without finding a working solution, the system of the disclosure may then offer the user the choice to either extend the time to complete the goal or specify a less ambitious health goal.

An embedded biophysical neural network of the system of the disclosure may rely on external feedback, which is handled by sensory neurons in the outer network layer. These sensory neurons receive relevant data at computing device 10 as input (e.g., physiological recordings provided by patient P such as ECG, respiration, body temperature). Relevant data features may then be extracted, and the resulting information may be transduced to a neuronal firing rate (i.e. action potential frequency) that propagates to the neurocomputational layers via simulated neurotransmission. This “ascending” sensory feedback may be received by a neuronal unit that combines the input signal with relevant feed-forward information (e.g. anticipated motor output) and re-distributes the result to corresponding neurocomputational network components, similar to the neurobiological role of the somatosensory cortex. The neurocomputational network layer may utilize this integrated signal to perform ongoing neurocomputational processes (e.g., received reward would inform the reinforcement learning process in the basal ganglia, and barometric feedback would inform the autonomic vascular control process in the brainstem). The neurocomputational layer may produce various physiological output (e.g., respiration, which is controlled via phrenic nerve motor output to the diaphragm).

By way of example and not limitation, a narrative description of a hypothetical setup of a profile, biophysical simulation, health network graph, and advisement for patient P may assist one skilled in the art to further understand the system, methods, and benefits of the system and method of the disclosure. At computing device 10, patient P may first input preliminary health information. This may include sex, age, height, and weight. Additional information may include data and/or diagnoses obtained via a psychological and/or medical survey. Patient P may then select or create a qualitative or quantitative wellness goal. An example of a qualitative goal may include simply “improve cardiovascular fitness” and a quantitative goal may include “decrease resting heart rate by 6 beats per minute”. Upon selection, patient P may then select an estimated timeframe to complete (e.g., 10 weeks). Then, a 24-hour baseline may be obtained through a variety of known means. These include things as invasive and sophisticated as inpatient monitoring via an EKG system or simply counting beats over 60-seconds using a wrist or neck pulse method at a regimented time throughout the day. It may also include the use of devices such as fitness wearables capable of measuring, recording, and transmitting such data. Other data measured, such as blood pressure, blood oxygen, sleep patterns, etc. may be measured and included in a baseline to increase the resolution of the biophysical simulation environment. Having obtained a 24-hour baseline of data, computing device 10 may then process and extract relevant statistical features (e.g., average heart and respiration rate). Using the extracted features, the biophysical network parameters obtained using the relevant calculations as described above may then be tuned via maximum likelihood until the model replicates the user's measured physiological output. This procedure would result in a quantitative estimate of the posterior parameter distribution, including the Hessian-approximated covariance matrix. Then, the “wellness timeframe” (i.e., the amount of time to accomplish the specified wellness goal) and the corresponding “wellness actions” would be predicted via iterative perturbation analysis. Small perturbations, relative to the data obtained or predicted using derived covariance approximation may then be applied to the relevant biophysical parameters. In each iteration, the simulated model output can be compared to the user goal. The corresponding parameter values which may be recorded if the result is significantly closer to the specified health goal. This process can be repeated, each time starting with the previous best parameter estimate, until the specified user health goal is accomplished in the resulting simulation. Outputs and/or advisements may include the predicted “wellness actions” which correspond to the optimal parameter values that were recorded during the iterative perturbation analysis procedure. These “wellness actions” may be used to define the recommended wellness plan. Additionally, a “wellness timeframe” may be computed using the cumulative sum of simulation time for each of the corresponding “wellness actions.”

By providing a simplified process of a closed vagal and sympathetic model, this narrative may be even better understood, and can be understood as optimizing the biophysical model for a respiratory-modulated innervation of the heart—a commonly understood biophysical phenomenon. Using a cardiorespiratory closed-loop model with the calculations provided above, a simplified model incorporating vagal and sympathetic (respiratory-modulated) innervation of the heart may be established. The baseline heart rate R0 may be computed from the user data as outlined above. The sympathetic and vagal inputs can be described by neuronal activation functions, whose parameters are statistically inferred from the measured heart rate variability using these calculations. Similarly, neuronal control of respiration is also described by the system of equations above. R1 is the activation of an expiratory neuronal population, and R2 is the inspiratory population. Statistical inference of these biophysical parameters (e.g., τ1, the expiration time constant) can therefore be used to quantify the relative importance of each health-relevant biophysical mechanism, which can be used to further optimize the biophysics simulation of the system of the disclosure, and the corresponding advisement. Using this optimized closed loop, a virtual assistant may create a wellness plan, personalized based on the user input from patient P, measurements of biological data from patient P, and calculated inferences. Having used various measurements, user entries, and calculations to establish an optimized biophysical simulation of a relevant organ or biological system of patient P, a personalized wellness plan may be assembled based on the user qualitative or quantitative goal. The system may be further utilized to adapt based on the performance of patient P as it relates to the goals established, new goals, inability to reach a goal, ease of reaching the goal, the like and/or combinations thereof. Other advantages include incorporation of various risks, comorbidities, atypical health factors, the like and/or combinations thereof with regard to both user input by patient P (e.g., heart condition), or via detection due to variance from expectations in biophysical network outputs.

Various features of the proposed system and method, and the underlying artificial intelligence and modelling approaches may further increase the utility of the proposed system and method. These include agent-based modelling, reinforcement learning, rate-based model of representation, and cardiovascular modelling. The artificial intelligence may be designed as an agent-based model that explores its environment (embedded biophysical network) by generating several candidate solutions, then selecting an optimal set via maximum likelihood estimation. Learning may occur within biophysical network components (e.g., cortex, basal ganglia). Thus, the artificial intelligence may replicate using machine learning the biophysical features of decision-making processes such as exploration, reward interpretation, and action selection. By training the artificial intelligence system to use machine learning to approximate the temporal dynamics (e.g., ionic time scales) of the neuronal processes that underlie executive control, decision making, and learning, patient P may be better influenced to include the recommendations and advisements of the system of the disclosure in their daily life and routines. The biophysical network may include functional models of dopaminergic reinforcement learning in the basal ganglia using reinforcement learning within the artificial intelligence system. The reinforcement learning model incorporates a mathematical description of striatal cholinergic interneurons. This may provide insight into the physiological mechanisms (e.g., activation time constants, ion channels, neurotransmitters) that determine learning preferences/performance. By using simplified models of neuronal control of respiration, the artificial intelligence system of the disclosure may replicate the functional dynamics of respiratory pattern-generation (e.g., inspiration/expiration phases, temporal dynamics of lung tidal volume). This respiration model may be designed as a simplified closed-loop cardiorespiratory neuronal circuitry as described above, which could be expanded for a more detailed approximation (e.g., incorporate the baroreflex, etc.). By working on and using machine learning to optimize the influence and approximation of the biophysical simulation, the artificial intelligence system may then train to extrapolate onto other systems outside of the closed loop, either based on new inputs or through inferences from existing data and calculations. A cardiovascular model may produce simulated cardiovascular output, incorporating vagal and sympathetic innervation of the heart and vasculature. The model describes blood pressure dynamics, and may replicate the physiology that determines cardiac output and VO2 max.

Another example of a simplified closed or semi-closed loop model may be an appetite model. This model may replicate the functional network-level dynamics of appetite. Importantly, the model may replicate the inverse relationship between food intake and working memory function. Studies have shown that working memory can be disrupted via high-calorie food intake. Reciprocally, amnesia patients have been shown to eat immediately after a large meal This phenomenon is hypothesized to be involved in the development of binge-eating disorders. This model could yield valuable insights into the temporal dynamics of appetite. This understanding may be particularly useful for the study and treatment of binge-eating disorders. By incorporating this model, a virtual assistant of the disclosure may produce personalized diet plans using a computational approach with the appetite calculations described in detail above. The proposed model may produce an appetite control signal that essentially represents the neurochemical by numerical integration of the nonlinear differential equations described above. Using those equations, the system of the disclosure may oscillate to produce action-potential-like spikes in E(t). Each spike may represent a meal. The size of each meal (MS) may represent the amplitude of a corresponding spike. The time between spikes is the inter-meal interval (IMI), which indicates how often a meal will be consumed. H(t) may be understood to dynamically influence the size of each meal. The satiety variable S(t) may reduce the frequency of meals. Synaptic inputs (IM(t), IH(t), IE(t), IS(t)) from external neuronal populations (e.g., cortex) may affect the intra-hypothalamic dynamics in the model. By optimizing meal size, inter-meal intervals, and other inputs as recommendations or advisements to increase satiety, the artificial intelligence system of the disclosure may further use machine learning to influence the eating behavior of patient P.

In the specification and/or figures, typical embodiments of the disclosure have been disclosed. The present disclosure is not limited to such exemplary embodiments. The use of the term “and/or” includes any and all combinations of one or more of the associated listed items. The figures are schematic representations and so are not necessarily drawn to scale. Unless otherwise noted, specific terms have been used in a generic and descriptive sense and not for purposes of limitation.

The foregoing description and drawings comprise illustrative embodiments. Having thus described exemplary embodiments, it should be noted by those skilled in the art that the within disclosures are exemplary only, and that various other alternatives, adaptations, and modifications may be made within the scope of the present disclosure. Merely listing or numbering the steps of a method in a certain order does not constitute any limitation on the order of the steps of that method. Many modifications and other embodiments will come to mind to one skilled in the art to which this disclosure pertains having the benefit of the teachings presented in the foregoing descriptions and the associated drawings. Although specific terms may be employed herein, they are used in a generic and descriptive sense only and not for purposes of limitation. Accordingly, the present disclosure is not limited to the specific embodiments illustrated herein but is limited only by the following claims.

1. A biophysics simulation system for improving the health of a patient comprising:
a computing device having a processor and a non-transitory computer readable medium connected to a sensor, said sensor is configured to detect a health information of the patient, said health information is relevant to a plurality of organs of the patient;
said non-transitory computer readable medium having there installed a program capable of simulating a biophysical environment of the patient;
a display capable of displaying a user interface, the user interface capable of communicating to the patient a health network graph; and
an executable artificial intelligence installed thereon said non-transitory computer readable medium capable of replicating neurocomputational decision making, thereby producing and updating a personalized wellness plan for the patient based upon the patient's unique health-relevant physiology and the patient's selection of a fitness goal;
wherein the health network graph is a visual representation of said plurality of organs of the patient, each of said plurality of organs represented as a node, and a plurality of connections among said plurality of organs represented as an edge of said health network graph and the fitness goal is an improvement of a quantitative measure of an at least one of said plurality of organs of the patient.

2. The system of claim 1, wherein the computing device is a wearable fitness tracker.

3. The system of claim 1, wherein the computing device is a smartphone.

4. The system of claim 1, wherein the health information that the sensor is configured to detect is an at least one biophysical data from a group of biophysical data, the group consisting of GPS, heart rate, blood pressure, blood oxygen, body temperature, blood glucose, and respiration rate.

5. The system of claim 1, wherein the executable artificial intelligence installed thereon said non-transitory computer readable medium is further configured to provide a health advisement to the patient.

6. The system of claim 2, wherein the wearable fitness tracker is capable of detecting a heart rate, an acceleration, and an altitude.

7. The system of claim 6, wherein in coordination with the executable artificial intelligence installed thereon said non-transitory computer readable medium, the computing device is configured to track the patient's adherence to the wellness plan.

8. The system of claim 7, wherein the computing device is further configured to make recommendations to the patient, via the display, based upon whether the patient has adhered to the wellness plan.

9. The system of claim 1, wherein the executable artificial intelligence is configured to optimize a parameter on the health network graph.

10. A method for generating a biophysics simulation for improving the health of a patient comprising:
on a computing device having a processor, a non-transitory computer readable medium, and a display, the computing device connected to a sensor capable of detecting a health information of the patient:
storing on said non-transitory computer readable medium a baseline health information;
collecting an at least one health data measurement from the patient via said sensor;
creating a biophysical simulation environment via said processor;
displaying a health network graph to the patient via said display, said health network graph, said health network graph visually representing a plurality of organs of the patient;
prompting the patient to select an organ from said plurality of organs of the patient for an improvement; and
displaying in detail a set of calculations relevant to said organ selected by the patient, based upon said baseline health information, said at least one health data measurement, and said biophysical simulation environment.

11. The method of claim 10, wherein the health network graph is a visual representation of said plurality of organs of the patient, each of said plurality of organs represented as a node, and a plurality of connections among said plurality of organs represented as an edge of said health network graph

12. The method of claim 10, wherein the computing device is a wearable fitness tracker.

13. The method of claim 10, wherein the computing device is a smartphone.

14. The method of claim 10, wherein the health information that the sensor is configured to detect is an at least one biophysical data from a group of biophysical data, the group consisting of GPS, heart rate, blood pressure, blood oxygen, body temperature, blood glucose, and respiration rate.

15. The method of claim 11, further comprising the step of optimizing the health network graph via an executable artificial intelligence installed thereon said non-transitory computer readable medium.

16. The method of claim 15, further comprising the step of prompting the user to select a fitness goal.

17. The method of claim 16, further comprising the step of generating a personalized wellness plan for the patient based upon the patient's unique health-relevant physiology and the patient's selection of the fitness goal.

18. The method of claim 17, further comprising the step of re-optimizing the health network graph via said executable artificial intelligence installed thereon said non-transitory readable medium based upon the patient's selection of the fitness goal.

19. The method of claim 17, further comprising the step of displaying said personalized wellness plan to the patient via the display.

20. The method of claim 15, wherein the step of creating the biophysical simulation environment is performed via the executable artificial intelligence installed thereon said non-transitory computer readable medium.
